(19) United States
(12) Patent Application Publication
(10) Pub. No.: US 2026/0048239 A1
(43) Pub. Date: Feb. 19, 2026

(54) COVER DRESSING, KIT, AND METHOD OF USE

(71) Applicant: Solventum Intellectual Properties Company, Maplewood, MN (US)

(72) Inventors: James M. Sieracki, Plymouth, MN (US); Kristopher E. Hunter, Woodbury, MN (US); Krystal J. Scheibel, Minneapolis, MN (US)

(21) Appl. No.: 18/996,154

(22) PCT Filed: Jul. 11, 2023
(86) PCT No.: PCT/IB2023/057122

§ 371 Date: Jan. 17, 2025

Related U.S. Application Data
(60) Provisional application No. 63/392,925, filed on Jul. 28, 2022.

(51) Int. Cl. A61M 25/02

(52) U.S. Cl. CPC A61M 25/02; A61M 2025/0266

(57) ABSTRACT

A cover dressing includes a backing including first and second major surfaces surrounded by a backing perimeter. The first major surface of the backing defines a dressing plane. The cover dressing further includes a cover film attached to the backing and including first and second major surfaces surrounded by a film perimeter. The cover film defines a first film portion extending beyond a backing perimeter and a second film portion at least partially coextensive with the backing, such that a film perimeter corresponding to the first film portion is at least partially spaced apart from the backing perimeter in the dressing plane. The cover dressing further includes a first adhesive layer at least partially disposed on at least one of the backing and the cover film. The first adhesive layer defines a first adhesive portion extending beyond the backing perimeter in the dressing plane.

### TECHNICAL FIELD

The present disclosure relates generally to cover dressings that are used to cover an insertion site, and particularly to a cover dressing for use with a medical device insertable through a skin of a patient, a kit including a pair of cover dressings, and a method of using the kit.

### BACKGROUND

Medical devices that include a tube or a cannula may be used for various purposes, such as feeding, air supply, liquid removal, and/or administering medications and fluids to a user. Such medical devices include, for example, central venous catheters, arterial catheters, drain tubes, and the like. The cannula of the medical devices may be inserted through a skin of a patient at an insertion site. The medical devices are typically secured to the patient (e.g., on the skin of the patient) by a securement device.

After insertion of the cannula through the skin of the patient, the insertion site may need to be covered by a dressing in order to protect the insertion site from infection. The dressing may provide a sterile barrier that keeps dirt, bacteria, and moisture away from the insertion site, thereby decreasing a risk of infection at the insertion site.

However, conventional dressings may need to be prematurely replaced due to adherence performance issues (e.g., dressing edge lift/roll) and/or excessive soiling (e.g., due to moisture or external material accessing the insertion site through channels in the conventional dressings). The risk of infection may increase each time the conventional dressings are removed from the insertion site for replacement purposes.

Therefore, there is a need for a cover dressing that provides an improved comfort to the patient during use, improved barrier properties, and an improved wear-time in order to reduce or prevent the need of premature replacement of the cover dressing.

### SUMMARY

In a first aspect, the present disclosure provides a cover dressing. The cover dressing includes a backing. The backing includes a first major surface and a second major surface opposing the first major surface. The backing further includes a backing perimeter surrounding the first major surface and the second major surface of the backing. The first major surface of the backing defines a dressing plane. The cover dressing further includes a cover film attached to the backing. The cover film includes a first major surface and a second major surface opposing the first major surface of the cover film. The second major surface of the cover film faces the first major surface of the backing. The cover film further includes a film perimeter surrounding the first major surface and the second major surface of the cover film. The cover film defines a first film portion extending beyond the backing perimeter and a second film portion at least partially coextensive with the backing, such that the film perimeter corresponding to the first film portion is at least partially spaced apart from the backing perimeter in the dressing plane. The cover dressing further includes a first adhesive layer at least partially disposed on at least one of the backing and the cover film. The first adhesive layer defines a first adhesive portion extending beyond the backing perimeter in the dressing plane. The first adhesive portion of the first adhesive layer is configured to at least partially bond with the skin of the patient.

In a second aspect, the present disclosure provides a kit for use with a medical device insertable through a skin of a patient at an insertion site. The kit includes a pair of cover dressings according to the first aspect.

In a third aspect, the present disclosure provides a kit for use with a medical device insertable through a skin of a patient at an insertion site. The kit includes a first cover dressing and a second cover dressing. Each of the first cover dressing and the second cover dressing includes a backing. The backing includes a first major surface, a second major surface opposing the first major surface, and a backing perimeter surrounding the first major surface and the second major surface of the backing. The first major surface of the backing defines a dressing plane. Each of the first cover dressing and the second cover dressing further includes a cover film. The cover film includes a first major surface, a second major surface opposing the first major surface and facing the first major surface of the backing, and a film perimeter surrounding the first major surface and the second major surface of the cover film. The cover film defines a first film portion extending beyond the backing perimeter and a second film portion at least partially coextensive with the backing, such that the film perimeter corresponding to the first film portion is at least partially spaced apart from the backing perimeter in the dressing plane. Each of the first cover dressing and the second cover dressing further includes a first adhesive layer partially disposed between the first major surface of the backing and the second major surface of the cover film. The first adhesive layer is further at least partially disposed on the second major surface of the cover film corresponding to the first film portion. The first adhesive layer is configured to at least partially bond with the skin of the user. Each of the first cover dressing and the second cover dressing further includes a second adhesive layer at least partially disposed on the second major surface of the backing. The second adhesive layer is configured to at least partially bond with the medical device.

In a fourth aspect, the present disclosure provides a method of using the kit of the third aspect. The method includes at least partially bonding the first adhesive layer of the first cover dressing to the skin of the patient, such that at least a portion of the backing perimeter of the first cover dressing is disposed adjacent to the insertion site. The method further includes rotating the medical device relative to the insertion site, such that the first cover dressing is folded and the second major surface of the backing faces away from the skin. The method further includes at least partially bonding the first adhesive layer of the second cover dressing to the skin of the patient, such that at least a portion of the backing perimeter of the second cover dressing is disposed adjacent to the insertion site, and the insertion site is together covered by the first cover dressing and the second cover dressing. The method further includes at least partially bonding the second adhesive layer of the first cover dressing and the second adhesive layer of the second cover dressing to the medical device and to each other, such that the medical device is at least partially disposed between and secured to the first cover dressing and the second cover dressing.

The details of one or more examples of the disclosure are set forth in the accompanying drawings and the description below. Other features, objects, and advantages of the disclosure will be apparent from the description and drawings, and from the claims.

### BRIEF DESCRIPTION OF THE DRAWINGS

Exemplary embodiments disclosed herein may be more completely understood in consideration of the following detailed description in connection with the following figures. The figures are not necessarily drawn to scale. In particular, thicknesses of certain layers in proportion to certain other items are exaggerated for ease of illustration and clarity purposes. Like numbers used in the figures refer to like components. However, it will be understood that the use of a number to refer to a component in a given figure is not intended to limit the component in another figure labeled with the same number.

FIG. 1 is a schematic perspective view of a medical device inserted through a skin of a patient;

FIG. 2A is a schematic cross-sectional view of a cover dressing according to an embodiment of the present disclosure;

FIG. 2B is a schematic exploded cross-sectional view of the cover dressing of FIG. 2A with some elements not shown according to an embodiment of the present disclosure;

FIG. 2C is a schematic top view of the cover dressing with some elements not shown according to an embodiment of the present disclosure;

FIG. 3 is a schematic bottom view of the cover dressing with some elements not shown according to an embodiment of the present disclosure;

FIG. 4 is a schematic cross-sectional view of a cover dressing according to another embodiment of the present disclosure;

FIG. 5A is a schematic cross-sectional view of a cover dressing according to another embodiment of the present disclosure;

FIG. 5B is a schematic cross-sectional view of a cover dressing according to another embodiment of the present disclosure;

FIG. 6A is a schematic cross-sectional view of a cover dressing according to another embodiment of the present disclosure;

FIG. 6B is a schematic cross-sectional view of a cover dressing according to another embodiment of the present disclosure;

FIG. 7 is a schematic block diagram of a kit according to an embodiment of the present disclosure;

FIG. 8 is a flowchart depicting various steps of a method of using the kit of FIG. 7 according to an embodiment of the present disclosure; and

FIGS. 9A-9G schematically illustrate the method of FIG. 8 according to an embodiment of the present disclosure.

### DETAILED DESCRIPTION

In the following description, reference is made to the accompanying figures that form a part thereof and in which various embodiments are shown by way of illustration. It is to be understood that other embodiments are contemplated and may be made without departing from the scope or spirit of the present disclosure. The following detailed description, therefore, is not to be taken in a limiting sense.

In the following disclosure, the following definitions are adopted.

As recited herein, all numbers should be considered modified by the term “about”. As used herein, “a,” “an,” “the,” “at least one,” and “one or more” are used interchangeably.

As used herein as a modifier to a property or attribute, the term “generally”, unless otherwise specifically defined, means that the property or attribute would be readily recognizable by a person of ordinary skill but without requiring absolute precision or a perfect match (e.g., within +/−20 % for quantifiable properties).

The term “substantially”, unless otherwise specifically defined, means to a high degree of approximation (e.g., within +/−10% for quantifiable properties) but again without requiring absolute precision or a perfect match.

The term “about”, unless otherwise specifically defined, means to a high degree of approximation (e.g., within +/−5% for quantifiable properties) but again without requiring absolute precision or a perfect match.

Terms such as same, equal, uniform, constant, strictly, and the like, are understood to be within the usual tolerances or measuring error applicable to the particular circumstance rather than requiring absolute precision or a perfect match.

As used herein, the terms “first” and “second” are used as identifiers. Therefore, such terms should not be construed as limiting of this disclosure. The terms “first” and “second” when used in conjunction with a feature or an element can be interchanged throughout the embodiments of this disclosure.

As used herein, when a first material is termed as “similar” to a second material, at least 90 weight % of the first and second materials are identical and any variation between the first and second materials comprises less than about 10 weight % of each of the first and second materials.

As used herein, “at least one of A and B” should be understood to mean “only A, only B, or both A and B”.

Unless specified or limited otherwise, the terms “attached,” “connected,” “coupled,” and variations thereof, are used broadly and encompass both direct and indirect attachments, connections, and couplings.

As used herein, the term “adjacent” refers to elements that are in proximity to each other, usually in contact with each other, but may have intervening elements between them.

As used herein, the term “configured to” is at least as restrictive as the term “adapted to” and requires actual design intention to perform the specified function rather than mere physical capability of performing such a function.

As used herein, the term “insertion site” refers to any site or region of a subject into which a tube or a cannula is intended to be inserted.

As used herein, the term “layer” refers to a thickness of a material or blend of materials. Layers may be continuous or discontinuous.

As used herein, the term “adhesive layer” or “tie layer” refers to a layer of adhesive material disposed on one or more layers to promote an adhesion of the one or more layers to each other or to another surface. Adhesive layers may be patterned.

As used herein, the term “perimeter” refers to a border, a circumference, or an outer boundary of a layer. It may be noted that a perimeter may be of any shape, such as, but not limited to, triangular, rectangular, octagonal, square, circular, oval, trapezoidal, pentagonal, hexagonal, and so forth.

As used herein, the term “coextensive” refers to a relationship between a first layer and a second layer where a length and/or width of the first layer is substantially equal to a respective length and/or width of the second layer.

The present disclosure relates to a cover dressing. The cover dressing includes a backing. The backing includes a first major surface and a second major surface opposing the first major surface. The backing further includes a backing perimeter surrounding the first major surface and the second major surface of the backing. The first major surface of the backing defines a dressing plane. The cover dressing further includes a cover film attached to the backing. The cover film includes a first major surface and a second major surface opposing the first major surface of the cover film. The second major surface of the cover film faces the first major surface of the backing. The cover film further includes a film perimeter surrounding the first major surface and the second major surface of the cover film. The cover film defines a first film portion extending beyond the backing perimeter and a second film portion at least partially coextensive with the backing, such that the film perimeter corresponding to the first film portion is at least partially spaced apart from the backing perimeter in the dressing plane. The cover dressing further includes a first adhesive layer at least partially disposed on at least one of the backing and the cover film. The first adhesive layer defines a first adhesive portion extending beyond the backing perimeter in the dressing plane. The first adhesive portion of the first adhesive layer is configured to at least partially bond with the skin of the patient.

The cover dressing of the present disclosure may be suitable for use with a medical device insertable through a skin of a patient. The cover dressing may be used, for example, to cover and protect an insertion site, i.e., a site at which a tube or a cannula of the medical device is inserted through the skin of the patient.

The cover dressing may protect the insertion site from dirt, bacteria, and moisture. Specifically, the cover film may provide barrier properties that protects the insertion site from dirt, bacteria, and moisture. The cover film may further be conformable and comfortable to wear for a long period of time. At least of a portion of the cover film may preferably be optically transparent, thereby allowing inspection of the insertion site without needing to remove the cover dressing from the skin of the patient. Further, the backing may provide mechanical stiffness to the cover dressing while allowing conformability to the skin of the patient.

The first adhesive layer may partially adhere the cover dressing to the skin of the patient. The first adhesive layer may include a skin compatible adhesive, and may preferably include an antimicrobial agent. The first adhesive layer may further partially adhere the cover dressing to the medical device. Specifically, the first adhesive layer may partially adhere the cover dressing to a cannula or a tube of the medical device.

In some cases, the cover dressing may further include a second adhesive layer that at least partially adheres the cover dressing to the medical device. The second adhesive layer may partially adhere the cover dressing to the cannula or the tube of the medical device. The second adhesive of the second adhesive layer may preferably include a repositionable adhesive, such as those including silicone.

In some cases, a pair of cover dressings of the present disclosure may be used to protect the insertion site. In such cases, the pair of cover dressings may be partially adhered to the skin of the patient, and partially adhered to the medical device, and partially adhered to each other.

The cover dressing may be used together with a securement device that secures and stabilizes the medical device to the skin of the patient. The cover dressing may advantageously secure and stabilize at least a portion of the medical device (e.g., a cannula or a tube) at the injection portion.

The cover dressing of the present disclosure may therefore provide improved wear-time and comfort to the patient. This may reduce or prevent premature replacement of the cover dressing at the injection site, thereby reducing a risk of infection at the insertion site.

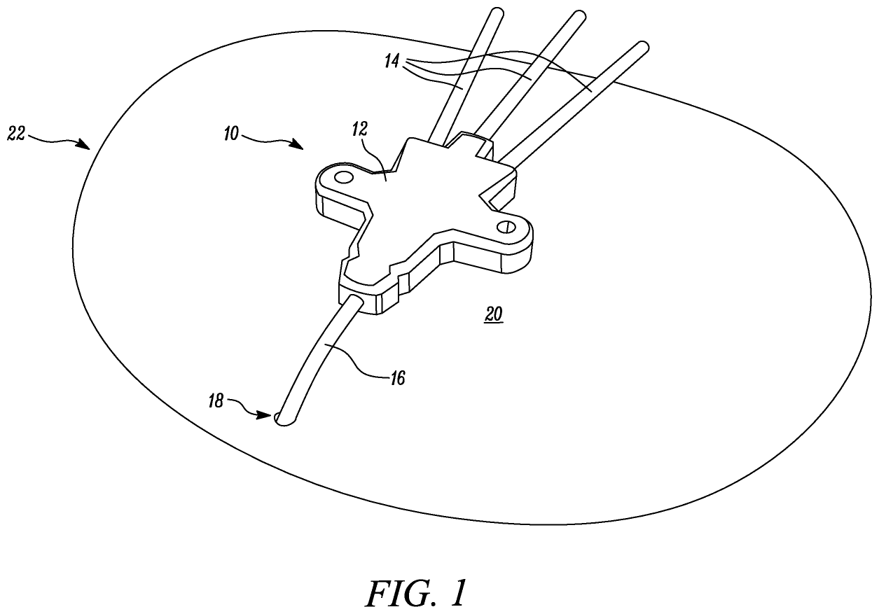

Referring now to the Figures, FIG. 1 illustrates a schematic perspective view of a medical device 10 insertable through a skin 20 of a patient 22. The medical device 10 may include a tube or a cannula 16. In FIG. 1, the cannula 16 of the medical device 10 is inserted through the skin 20 of the patient 22 at an insertion site 18.

The medical device 10 may further include one or more lumens 14 fluidly connected with the cannula 16. The one or more lumens 14 are partially shown in FIG. 1. The medical device 10 may further include a hub 12. The hub 12 may at least partially receive the one or more lumens 14 and the cannula 16.

The medical device 10 illustrated in FIG. 1 is a central venous catheter (CVC). Alternatively, the medical device 10 may be, for example, an arterial catheter, a drain tube, a peripherally inserted central catheter (PICC), and the like.

In some cases, the hub 12 may be secured and stabilized on the skin 20 of the patient 22 by a securement device (not shown). Furthermore, the insertion site 18 may be covered by a dressing (not shown in FIG. 1). The dressing may be intended to provide a sterile barrier that protects the insertion site 18 from dirt, bacteria, and moisture, thereby decreasing a risk of infection at the insertion site 18. In some cases, the dressing may further stabilize the cannula 16 relative to the insertion site 18.

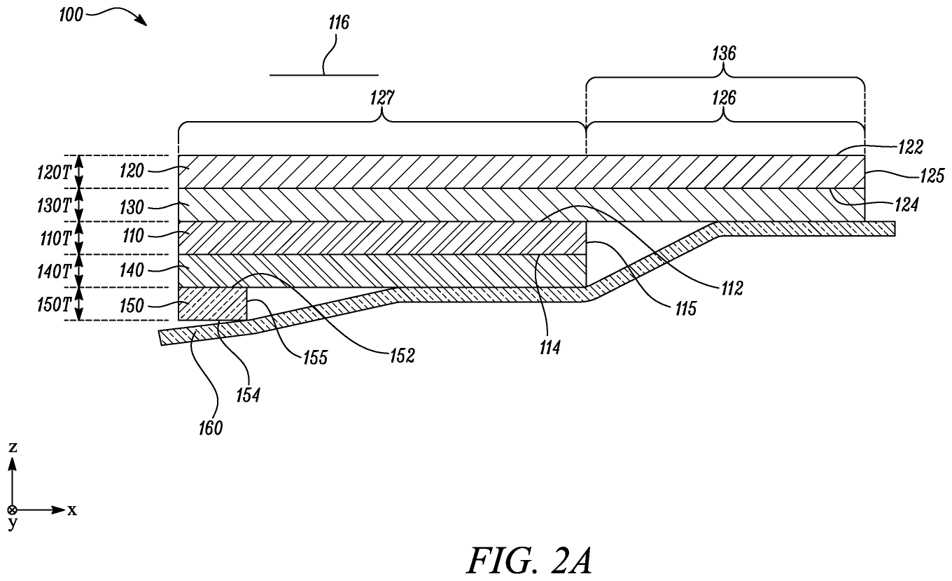

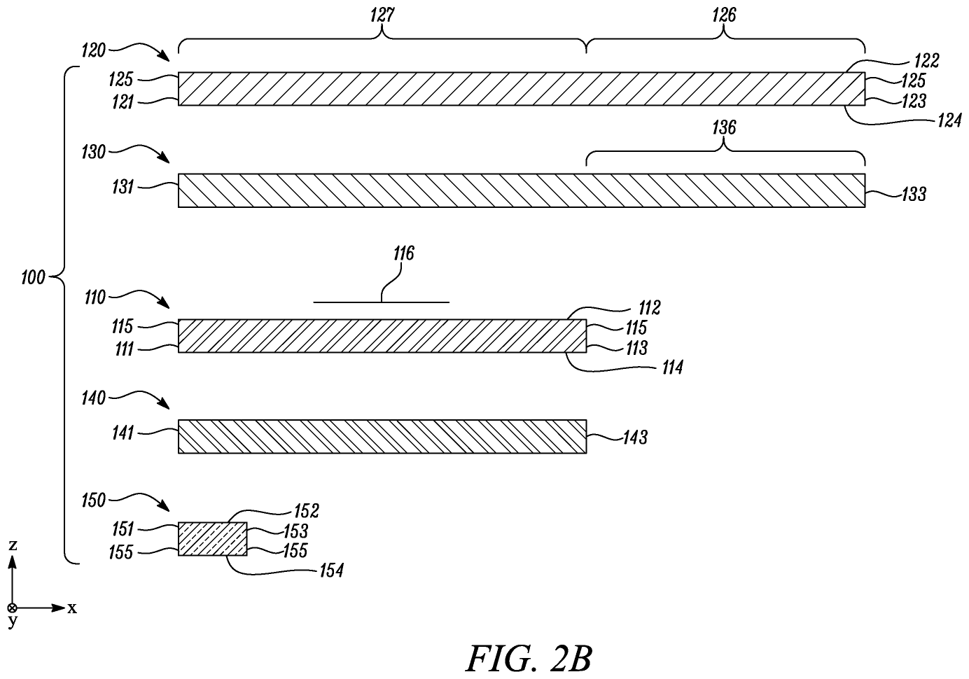

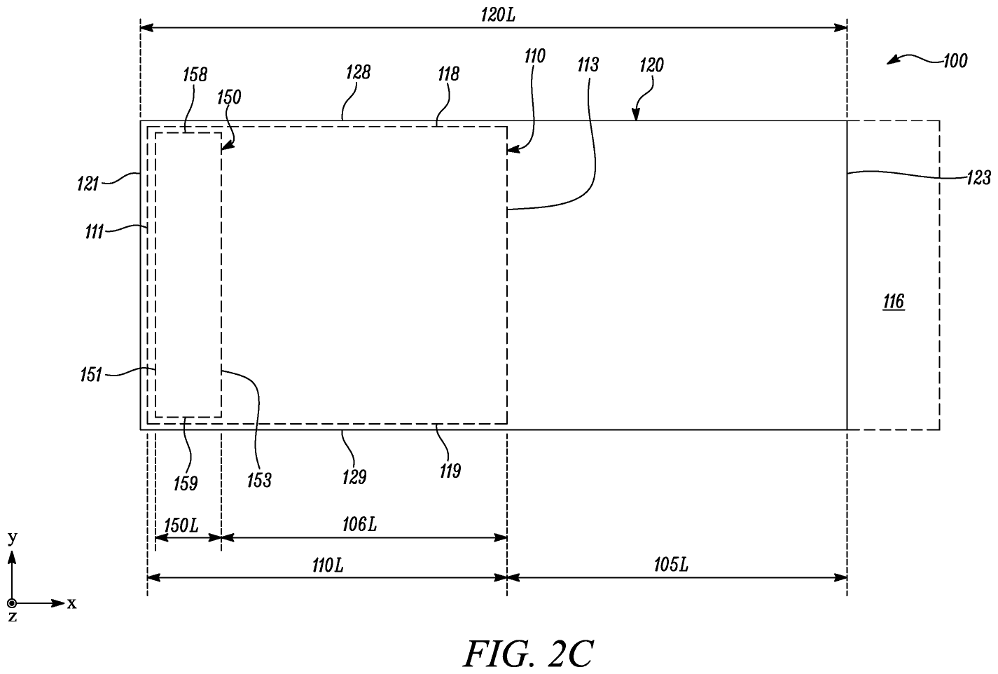

FIG. 2A illustrates a schematic cross-sectional view of a cover dressing 100 according to an embodiment of the present disclosure. The cover dressing 100 may be suitable for use with a medical device (e.g., the medical device 10 of FIG. 1) insertable through a skin of a patient (e.g., the skin 20 of the patient 22 of FIG. 1). FIG. 2B illustrates a schematic exploded cross-sectional view of the cover dressing 100.

The cover dressing 100 defines mutually orthogonal x, y, and z-axes. The x-axis is defined along a length of the cover dressing 100, while the y-axis is defined along a breadth of the cover dressing 100. The Z-axis is defined along a thickness of the cover dressing 100.

Referring to FIGS. 2A and 2B, the cover dressing 100 includes a backing 110. The backing 110 includes a first major surface 112 and a second major surface 114 opposing the first major surface 112. The backing 110 further includes a backing perimeter 115 surrounding the first major surface 112 and the second major surface 114 of the backing 110.

The first major surface 112 of the backing 110 defines a dressing plane 116. The dressing plane 116 is illustratively shown with a line in FIGS. 2A and 2B. The dressing plane 116 may be substantially parallel to the first major surface 112 of the backing 110. The dressing plane 116 may be substantially parallel to the x-y plane.

The backing 110 may include any material that provides mechanical stiffening to the cover dressing 100 while providing adequate flexibility to allow for conformability and comfortable wear. For example, the material of the backing 110 may be a film, a paper, or a fabric layer material, such as a woven, knitted, or nonwoven fabric. The material of the backing 110 may be elastic or stiff.

In some embodiments, the backing 110 includes a foam or a nonwoven material. For example, the backing 110 may include a woven, knitted, or nonwoven material. One example of a nonwoven material is a high strength nonwoven fabric available from E.I. Dupont de Nemours & Company of Wilmington, Delaware, under the trademark SONTARA. Other suitable nonwoven materials include, but are not limited to, a hydroentangled polyester fabric available from Vertac, a division of International Paper of Walpole, Minnesota. Another suitable nonwoven material is the nonwoven elastomeric web described in U.S. Pat. No. 5,230,701, which is incorporated herein by reference.

The backing 110 may be a high moisture vapor permeable film backing. U.S. Pat. No. 3,645,835 describes methods of making such films and methods for testing their permeability and is incorporated herein by reference. The backing 110 may have a single layer or multilayer construction. In some examples, a reinforcing material may be included with the backing 110. The reinforcing material may be as pliable as a thick adhesive or as stiff as a solid material (e.g., a paper or a film).

The backing 110 further includes a thickness 110T defined between the first major surface 112 and the second major surface 114. The thickness 110T of the backing 110 may be defined perpendicular to the dressing plane 116 (i.e., along the z-axis). In some embodiments, the thickness 110T of the backing 110 may be from 25 microns to 260 microns (i.e., about 1 mils to about 10 mils). In some embodiments, the thickness 110T of the backing 110 may be from 50 microns to 130 microns.

The cover dressing 100 further includes a cover film 120 attached to the backing 110. The cover film 120 may be directly (e.g., stitching, etc.) or indirectly attached (e.g., via adhesives) to the backing 110. In the illustrated embodiment of FIG. 2A, the cover film 120 is attached to the backing 110 via an adhesive layer, which will be described later in greater detail.

The cover film 120 includes a first major surface 122 and a second major surface 124 opposing the first major surface 122 of the cover film 120. The second major surface 124 of the cover film 120 faces the first major surface 112 of the backing 110. The cover film 120 further includes a film perimeter 125 surrounding the first major surface 122 and the second major surface 124 of the cover film 120.

The cover film 120 defines a first film portion 126 extending beyond the backing perimeter 115 and a second film portion 127 at least partially coextensive with the backing 110, such that the film perimeter 125 corresponding to the first film portion 126 is at least partially spaced apart from the backing perimeter 115 in the dressing plane 116. In other words, at least a portion of the cover film 120 may be non-coextensive with the backing 110. The cover film 120 may have greater dimensions than the backing 110 in the dressing plane 116 (or the x-y plane), such that the cover film 120 defines the first film portion 126 that extends beyond the backing perimeter 115 of the backing 110.

In some embodiments, the cover film 120 is optically transparent. In some embodiments, at least the first film portion 126 of the cover film 120 is optically transparent. As a result, the skin of the patient disposed underneath the first film portion 126 may be visible through the cover dressing 100. The skin disposed underneath the first film portion 126 may therefore be examined without having to remove the cover dressing 100 from the skin of the patient. In some examples, the insertion site (e.g., the insertion site 18 of FIG. 1) may be visible through the first film portion 126 and therefore may be examined without having to remove the cover dressing 100 from the skin of the patient. The cover film 120 being optically transparent may also facilitate positioning of the cover dressing 100 relative to the insertion site during application of the cover dressing 100.

The cover film 120 may preferably be impermeable to liquids but permeable to moisture vapor. Further, the cover film 120 may be conformable and resilient. For example, the cover film 120 may conform to nonplanar anatomical surfaces, such as the skin of the patient. In some examples, the cover film 120 may have an ultimate elongation greater than 200%. In some examples, the cover film 120 may have an ultimate elongation greater than 400%. Exemplary materials for the cover film 120 can be found, for example, in U.S. Pat. Nos. 5,088,483 and 5,160,315, which are incorporated herein by reference. In some examples, the cover film 120 may include elastomeric polyurethane, co-polyester, or polyether block amide films. Therefore, the cover film 120 may have desirable properties of conformability, high moisture vapor permeability, and transparency.

The cover film 120 includes a thickness 120T defined between the first major surface 122 and the second major surface 124. The thickness 120T of the cover film 120 may be defined perpendicular to the dressing plane 116 (i.e., along the z-axis). In some embodiments, the thickness 120T of the cover film 120 may be from 12 microns to 130 microns (i.e., about 0.5 mils to about 5 mils). In some embodiments, the thickness 120T of the cover film 120 may be from 25 microns to 100 microns.

In some embodiments, each of the backing 110 and the cover film 120 is continuous in the dressing plane 116 and is devoid of any through-openings. In other words, each of the backing 110 and the cover film 120 may be devoid of any macro-openings (e.g., openings having a maximum width greater than 1 millimeter). In some embodiments, each of the backing 110 and the cover film 120 may include micro-openings that can impart vapor permeability and be devoid of macro-openings. Each of the backing 110 and the cover film 120 may therefore be free of macroscopic elements, such as a window or a screen extending therethrough.

The cover dressing 100 further includes a first adhesive layer 130 at least partially disposed on at least one of the backing 110 and the cover film 120. The first adhesive layer 130 defines a first adhesive portion 136 extending beyond the backing perimeter 115 in the dressing plane 116. The first adhesive portion 136 of the first adhesive layer 130 is configured to at least partially bond with the skin of the patient (e.g., the skin 20 of the patient 22 of FIG. 1).

In the illustrated embodiment of FIG. 2A, the first adhesive layer 130 is partially disposed between the first major surface 112 of the backing 110 and the second major surface 124 of the cover film 120, such that the first adhesive portion 136 is at least partially coextensive with the first film portion 126 in the dressing plane 116. Specifically, in the illustrated embodiment of FIG. 2A, the first adhesive layer 130 is coextensive with the second major surface 124 of the cover film 120 in the dressing plane 116.

The first adhesive layer 130 may include an adhesive material that is suitable for application on the skin. The first adhesive layer 130 may include an optically clear adhesive (OCA). The first adhesive layer 130 may include, for example, a pressure-sensitive adhesive (PSA). The pressure sensitive adhesive may have a relatively high moisture vapor transmission rate to allow for moisture evaporation. Suitable pressure sensitive adhesives include those based on acrylates, urethane, hydrogels, hydrocolloids, block copolymers, silicones, rubber-based adhesives (including natural rubber, polyisoprene, polyisobutylene, butyl rubber etc.) as well as combinations of these adhesives. The adhesive component may contain tackifiers, plasticizers, rheology modifiers, and active components. The pressure sensitive adhesive may be reasonably skin compatible and “hypoallergenic”, such as the acrylate copolymers described in U.S. Pat. No. RE 24,906. Particularly useful is a 97:3 iso-octyl acrylate:acrylamide copolymer, as is 70:15:15 isooctyl acrylate:ethyleneoxide acrylate:acrylic acid terpolymer described in U.S. Pat. No. 4,737,410. Additional useful adhesives are described in U.S. Pat. Nos. 3,389,827; 4,112,213; 4,310,509; and 4,323,557. In some embodiments, the first adhesive layer 130 includes an antimicrobial agent. Inclusion of medicaments or the antimicrobial agent in the first adhesive layer 130 is contemplated, as described in U.S. Pat. Nos. 4,310,509 and 4,323,557.

In the illustrated embodiment of FIG. 2A, the first adhesive layer 130 is coextensive with the second major surface 124 of the cover film 120. Alternatively, the first adhesive layer 130 may be partially non-coextensive with the second major surface 124 of the cover film 120, with at least a portion of the first adhesive layer 130 disposed on the second major surface 124 of the cover film 120 corresponding to the first film portion 126. The first adhesive layer 130 may be substantially continuous or patterned, based on desired application attributes. The first adhesive layer 130 being patterned may advantageously improve the vapor transmission rate of the cover dressing 100.

The first adhesive layer 130 includes a thickness 130T. The thickness 130T of the first adhesive layer 130 may be defined perpendicular to the dressing plane 116 (i.e., along the z-axis). In some embodiments, the thickness 130T of the first adhesive layer 130 may be from 2.5 microns to 130 microns (i.e., about 0.1 mils to about 5 mils). In some embodiments, the thickness 130T of the first adhesive layer 130 may be from 5 microns to 50 microns.

In the illustrated embodiment of FIG. 2A, the cover dressing 100 further includes a second adhesive layer 140 at least partially disposed on the second major surface 114 of the backing 110. The second adhesive layer 140 may be configured to at least partially bond with the medical device (e.g., the medical device 10 of FIG. 1). Specifically, the second adhesive layer 140 may be configured to at least partially bond with a tube or a cannula of the medical device (e.g., the cannula 16 of the medical device 10 of FIG. 1).

The second adhesive layer 140 may include a repositionable adhesive. Preferably, the second adhesive may be easily removable from the medical device, such that the cover dressing 100 can be removed from the skin of the patient without having to remove the medical device from the insertion site. Furthermore, the second adhesive layer 140 may have a low cohesive strength at room temperature (e.g., at 27 degrees Celsius).

In some examples, the second adhesive layer 140 may include a pressure sensitive adhesive. In some embodiments, the second adhesive layer 140 includes silicone. Suitable silicone adhesives are disclosed in PCT Publications WO2010/056541 and WO2010/056543, the disclosures of which are incorporated herein by reference. In some embodiments, the second adhesive layer 140 may include a different adhesive than the first adhesive layer 130. In other words, the second adhesive layer 140 may include a different composition of adhesive than that of the first adhesive layer 130.

In the illustrated embodiment of FIG. 2A, the second adhesive layer 140 is coextensive with the second major surface 114 of the backing 110. Alternatively, the second adhesive layer 140 may be partially non-coextensive with the second major surface 114 of the backing 110. The second adhesive layer 140 may be substantially continuous or patterned, based on desired application attributes. In some cases, the second adhesive layer 140 may be discontinuous along a portion of the second major surface 114 of the backing 110.

The second adhesive layer 140 includes a thickness 140T. The thickness 140T of the second adhesive layer 140 may be defined perpendicular to the dressing plane 116 (i.e., along the z-axis). In some embodiments, the thickness 140T of the second adhesive layer 140 may be from 2.5 microns to 130 microns (i.e., about 0.1 mils to about 5 mils). In some embodiments, the thickness 140T of the second adhesive layer 140 may be from 5 microns to 50 microns.

In the illustrated embodiment of FIG. 2A, the cover dressing 100 further includes a tab 150. The tab 150 may include a first major surface 152 facing the second major surface 114 of the backing 110 and a second major surface 154 opposing the first major surface 152 of the tab 150. The tab 150 may further include a tab perimeter 155 surrounding the first major surface 152 and the second major surface 154 of the tab 150.

The tab 150 further includes a thickness 150T. The thickness 150T of the tab 150 may be defined between the first major surface 152 and the second major surface 154 of the tab 150. The thickness 150T of the tab 150 may be defined perpendicular to the dressing plane 116 (i.e., along the z-axis). In some embodiments, the thickness 150T of the tab 150 may be from 12 microns to 130 microns (i.e., about 0.5 mils to about 5 mils).

The tab 150 may provide a gripping region that is free of any adhesive, such as those of the first adhesive layer 130 and the second adhesive layer 140. Specifically, the second major surface 154 of the tab 150 may be free of any adhesive. The tab 150 may be gripped by a user to apply the cover dressing 100 on the skin of the patient. Specifically, the second major surface 154 of the tab 150 and the first major surface 122 of the cover film 120 may be grasped by the user to apply the cover dressing 100 on the skin of the patient. However, it may be noted that the tab 150 is optional. The tab 150 may be omitted from the cover dressing 100, for example, if the second adhesive layer 140 has a configuration that provides an adhesive-free region on the second major surface 114 of the backing 110. Such a configuration will be discussed later in greater detail.

In the illustrated embodiment of FIG. 2A, the second adhesive layer 140 is partially disposed between the second major surface 114 of the backing 110 and the first major surface 152 of the tab 150. Further, in the illustrated embodiment of FIG. 2A, each of the backing 110 and the second adhesive layer 140 extends beyond the tab perimeter 155, such that the backing perimeter 115 is at least partially spaced apart from the tab perimeter 155 in the dressing plane 116.

In some embodiments, an area of the first major surface 112 of the backing 110 is at most 60% of an area of the second major surface 124 of the cover film 120. In some embodiments, the area of the first major surface 112 of the backing 110 is at most 50%, at most 40%, or at most 30% of the area of the second major surface 124 of the cover film 120. The cover film 120 may therefore be greater in dimensions as compared to the backing 110. In some embodiments, an area of the second major surface 154 of the tab is at most 30% of an area of the second major surface 114 of the backing 110. The backing 110 may therefore be greater in dimensions as compared to the tab 150. The cover film 120, the backing 110, and the tab 150 may have sequentially decreasing dimensions in the dressing plane 116.

In the illustrated embodiment of FIG. 2A, the tab perimeter 155 is partially aligned with the backing perimeter 115 in the dressing plane 116. Moreover, in the illustrated embodiment of FIG. 2A, the backing perimeter 115 is partially aligned with the film perimeter 125 in the dressing plane 116. In other words, in the illustrated embodiment of FIG. 2A, the film perimeter 125, the backing perimeter 115, and the tab perimeter 155 are partially aligned with each other and partially misaligned with each other. For example, the film perimeter 125, the backing perimeter 115, and the tab perimeter 155 may be aligned at one end of the cover dressing 100 while being misaligned at an opposing end of the cover dressing 100.

In some embodiments, the cover dressing 100 further includes a release liner 160 at least partially and removably adhered to each of the first adhesive layer 130 and the second adhesive layer 140. The release liner 160 is not shown in FIG. 2B for illustrative purposes.

The release liner 160 may protect the first adhesive layer 130 and the second adhesive layer 140 from undesired contaminants, such as dust, debris, and the like, prior to application of the cover dressing 100 on the skin of the patient. A removal of the release liner 160 may expose at least respective portions of the first adhesive layer 130 and the second adhesive layer 140. In some other embodiments, the cover dressing 100 may include separate release liners for the first adhesive layer 130 and the second adhesive layer 140. Such configuration of release liners will be discussed later in greater detail.

FIG. 2C illustrates a schematic top view of the cover dressing 100 according to an embodiment of the present disclosure. The first adhesive layer 130, the second adhesive layer 140, and the release liner 160 that are shown in FIG. 2A are not shown in FIG. 2C for illustrative purposes. Furthermore, the backing 110 and the tab 150 are schematically shown with dashed lines in FIG. 2C for illustrative purposes.

The cover dressing 100 may have any suitable shape, such as circular, oval, rectangular, oblong, polygonal, and the like. In the illustrated embodiment of FIG. 2C, the cover dressing 100 has a substantially rectangular shape including a pair of transverse edges and a pair of longitudinal edges.

In the illustrated embodiment of FIG. 2C, the cover film 120 has a generally rectangular shape and includes a first transverse edge 121, a second transverse edge 123 opposite to the first transverse edge 121, a first longitudinal edge 128, and a second longitudinal edge 129 opposite to the first longitudinal edge 128. The first transverse edge 121 and the second transverse edge 123 may extend along the y-axis. The first longitudinal edge 128 and the second longitudinal edge 129 may extend along the x-axis.

The first transverse edge 121, the second transverse edge 123, the first longitudinal edge 128, and the second longitudinal edge 129 may collectively define the film perimeter 125 (shown in FIGS. 2A and 2B). The cover film 120 may define a film length 120L extending along the first longitudinal edge 128 and the second longitudinal edge 129 and between the first transverse edge 121 and the second transverse edge 123 of the cover film 120.

In the illustrated embodiment of FIG. 2C, the backing 110 has a substantially rectangular shape and includes a first transverse edge 111, a second transverse edge 113 opposite to the first transverse edge 111, a first longitudinal edge 118, and a second longitudinal edge 119 opposite to the first longitudinal edge 118. The first transverse edge 111 and the second transverse edge 113 may extend along the y-axis. The first longitudinal edge 118 and the second longitudinal edge 119 may extend along the x-axis. The first transverse edge 111, the second transverse edge 113, the first longitudinal edge 118, and the second longitudinal edge 119 may collectively define the backing perimeter 115 (shown in FIGS. 2A and 2B).

In the illustrated embodiment of FIG. 2C, the second transverse edge 113 of the backing 110 is spaced apart from the second transverse edge 123 of the cover film 120. Furthermore, in FIG. 2C, the first transverse edge 111, the first longitudinal edge 118, and the second longitudinal edge 119 of the backing 110 are shown as being slightly misaligned with the first transverse edge 121, the first longitudinal edge 128, and the second longitudinal edge 129, respectively, of the cover film 120 for illustrative purposes only, so that the respective first transverse edges can be distinguished from each other. It may be noted that the first transverse edge 111 of the backing 110 may or may not be aligned with the first transverse edge 121 of the cover film 120, the first longitudinal edge 118 of the backing 110 may or may not be aligned with the first longitudinal edge 128 of the cover film 120, and the second longitudinal edge 119 of the backing 110 may or may not be aligned with the second longitudinal edge 129 of the cover film 120.

The backing 110 may define a backing length 110L extending along the first longitudinal edge 118 and the second longitudinal edge 119 and between the first transverse edge 111 and the second transverse edge 113 of the backing 110. The backing length 110L may be less than the film length 120L. Specifically, the backing length 110L may be less than the film length 120L by a first length difference 105L. In other words, the first length difference 105L may be a difference in length between the film length 120L and the backing length 110L. The first length difference 105L may be, for example, greater than or equal to 50% of the film length 120L.

In the illustrated embodiment of FIG. 2C, the tab 150 has a substantially rectangular shape and includes a first transverse edge 151, a second transverse edge 153 opposite to the first transverse edge 151, a first longitudinal edge 158, and a second longitudinal edge 159 opposite to the first longitudinal edge 158. The first transverse edge 151 and the second transverse edge 153 may extend along the y-axis. The first longitudinal edge 158 and the second longitudinal edge 159 may extend along the x-axis. The first transverse edge 151, the second transverse edge 153, the first longitudinal edge 158, and the second longitudinal edge 159 may collectively define the tab perimeter 155 (shown in FIGS. 2A and 2B).

In the illustrated embodiment of FIG. 2C, the second transverse edge 153 of the tab 150 is spaced apart from the second transverse edge 113 of the backing 110. Furthermore, in FIG. 2C, the first transverse edge 151, the first longitudinal edge 158, and the second longitudinal edge 159 of the tab 150 are shown as being slightly misaligned with the first transverse edge 121, the first longitudinal edge 128, and the second longitudinal edge 129, respectively, of the cover film 120 for illustrative purposes only, so that the respective first transverse edges can be distinguished from each other. It may be noted that the first transverse edge 151 of the tab 150 may or may not be aligned with the first transverse edge 111 of the backing 110, the first longitudinal edge 158 of the tab 150 may or may not be aligned with the first longitudinal edge 118 of the backing 110, and the second longitudinal edge 159 of the tab 150 may or may not be aligned with the second longitudinal edge 119 of the backing 110. In the illustrated embodiment of FIGS. 2A and 2B, the first transverse edge 111 of the backing 110, the first transverse edge 121 of the cover film 120, and the first transverse edge 151 of the tab 150 are aligned with each other in the dressing plane 116 (shown by dashed lines in FIG. 2C).

The tab 150 may define a tab length 150L extending along the first longitudinal edge 158 and the second longitudinal edge 159 and between the first transverse edge 151 and the second transverse edge 153 of the tab 150. The tab length 150L may be less than the backing length 110L. Specifically, the tab length 150L may be less than the backing length 110L by a second length difference 106L. In other words, the second length difference 106L may be a difference in length between the backing length 110L and the tab length 150L. The second length difference 106L may be, for example, greater than or equal to 50% of the backing length 110L.

In the illustrated embodiment of FIG. 2B, the first adhesive layer 130 includes a first transverse edge 131 and a second transverse edge 133 opposite to the first transverse edge 131. Moreover, in the illustrated embodiment of FIG. 2B, the second adhesive layer 140 includes a first transverse edge 141 and a second transverse edge 143 opposite to the first transverse edge 141.

Referring now to FIGS. 2A-2C, in some embodiments, each of the backing 110, the cover film 120, the first adhesive layer 130, the second adhesive layer 140, and the tab 150 includes the respective first transverse edge 111, 121, 131, 141, 151 and the respective second transverse edge 113, 123, 133, 143, 153 opposite to the respective first transverse edge 111, 121, 131, 141, 151. Further, in some embodiments, the respective first transverse edges 111, 121, 131, 141, 151 of the backing 110, the cover film 120, the first adhesive layer 130, the second adhesive layer 140, and the tab 150 are aligned in the dressing plane 116. In other words, in some embodiments, the first transverse edge 111 of the backing 110, the first transverse edge 121 of the cover film 120, the first transverse edge 131 of the first adhesive layer 130, and the first transverse edge 141 of the second adhesive layer 140 are aligned with each other in the dressing plane 116. In some embodiments, the first transverse edge 151 of the tab 150 may also be aligned with the first transverse edge 111 of the backing 110, the first transverse edge 121 of the cover film 120, the first transverse edge 131 of the first adhesive layer 130, and the first transverse edge 141 of the second adhesive layer 140 in the dressing plane 116.

Further, in some embodiments, the respective second transverse edges 113, 123, 133, 143, 153 of the backing 110, the cover film 120, the first adhesive layer 130, the second adhesive layer 140, and the tab 150 are misaligned in the dressing plane 116. In other words, in some embodiments, the second transverse edge 113 of the backing 110, the second transverse edge 123 of the cover film 120, the second transverse edge 133 of the first adhesive layer 130, and the second transverse edge 143 of the second adhesive layer 140 are misaligned with each other in the dressing plane 116. In some embodiments, the second transverse edge 153 of the tab 150 may also be misaligned with the second transverse edge 113 of the backing 110, the second transverse edge 123 of the cover film 120, the second transverse edge 133 of the first adhesive layer 130, and the second transverse edge 144 of the second adhesive layer 140 in the dressing plane 116.

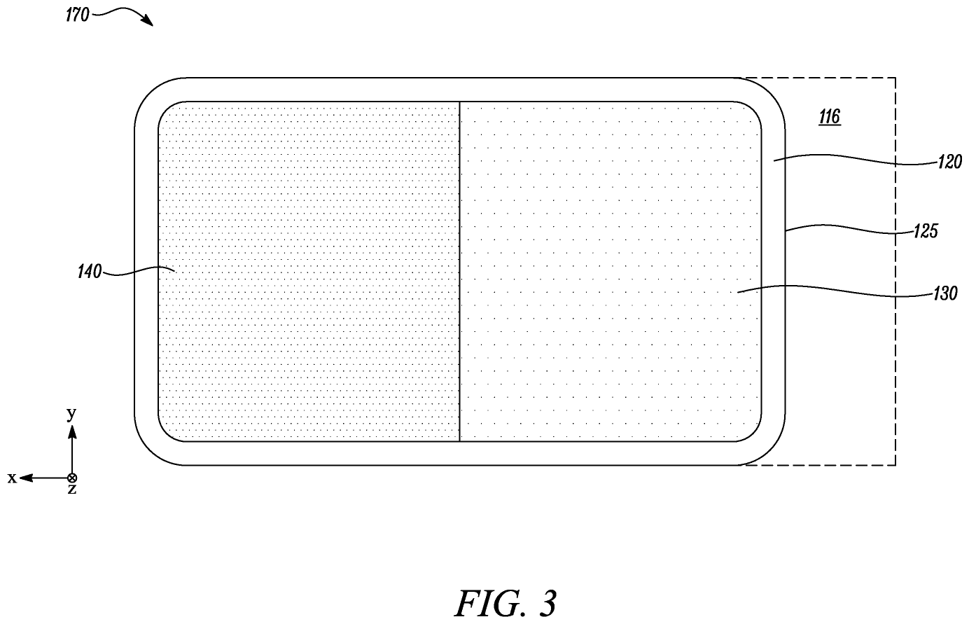

FIG. 3 illustrates schematic a bottom view of a cover dressing 170 according to another embodiment of the present disclosure. The cover dressing 170 is substantially similar to the cover dressing 100 of FIG. 2A, with like elements designated by like reference characters. The backing 110 and the tab 150 (both shown in FIG. 2A) are not shown in FIG. 3 for illustrative purposes. However, the cover dressing 170 has a different shape and a different configuration of the first adhesive layer 130 and the second adhesive layer 140 than that of the cover dressing 100 of FIG. 2A.

Specifically, in the illustrated embodiment of FIG. 3, the cover dressing 170 has a rectangular shape with rounded edges. In the illustrated embodiment, the cover film 120 has a rectangular shape with rounded edges. However, it may be noted that one or more of the cover film 120, the backing 110 (not shown in FIG. 3), the first adhesive layer 130, the second adhesive layer 140, and the tab 150 (not shown in FIG. 3) may have a rectangular shape with rounded edges.

Furthermore, in the illustrated embodiment of FIG. 3, each of the first adhesive layer 130 and the second adhesive layer 140 is spaced apart from the film perimeter 125 in the dressing plane 116 (i.e., the x-y plane in FIG. 3).

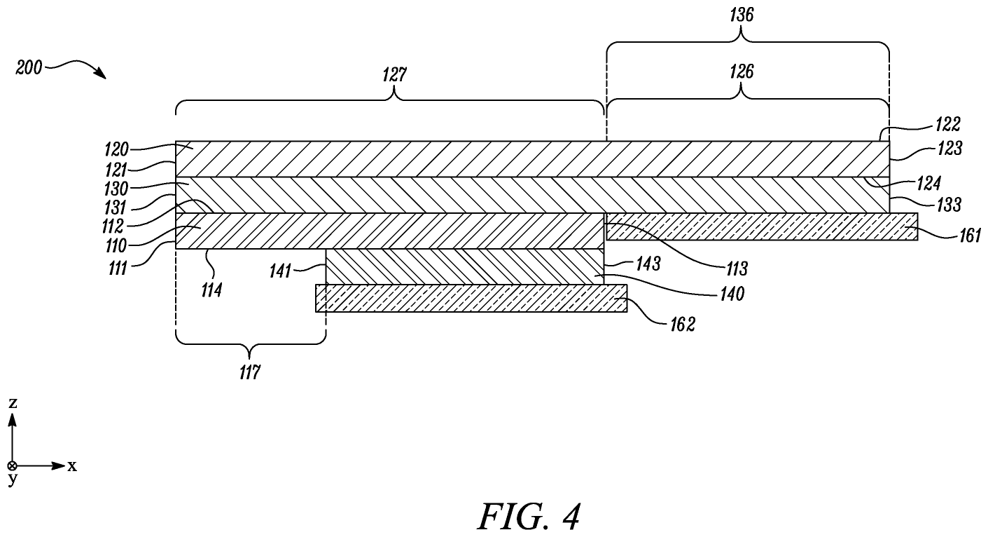

FIG. 4 illustrates a schematic cross-sectional view of a cover dressing 200 according to another embodiment of the present disclosure. The cover dressing 200 is similar to the cover dressing 100 of FIG. 2A, with like elements designated by like reference characters. However, the cover dressing 200 includes a different configuration of the second adhesive layer 140 as compared to the cover dressing 100 of FIG. 2A. As a result, the tab 150 of the cover dressing 100 is omitted in the cover dressing 200.

In the illustrated embodiment of FIG. 4, the second major surface 114 of the backing 110 includes an adhesive-free region 117 extending beyond the second adhesive layer 140. In the illustrated embodiment of FIG. 4, the first transverse edge 141 of the second adhesive layer 140 is spaced apart from the first transverse edge 111 of the backing 110, such that the adhesive-free region 117 is defined by the second major surface 114 of the backing 110.

The adhesive-free region 117 may be gripped by a user to apply the cover dressing 200 on the skin of the patient. The adhesive-free region 117 may offer similar functionality to that of the tab 150 of FIG. 2A. As a result, the adhesive-free region 117 may allow omission of the tab 150 (shown in FIG. 2A) from the cover dressing 200.

In the illustrated embodiment of FIG. 4, the cover dressing 200 further includes a first release liner 161 at least partially and removably adhered to the first adhesive layer 130. A removal of the first release liner 161 may expose at least a portion of the first adhesive layer 130. In the illustrated embodiment of FIG. 4, the cover dressing 200 further includes a second release liner 162 at least partially and removably adhered to the second adhesive layer 140. A removal of the second release liner 162 may expose at least a portion of the second adhesive layer 140. Therefore, the respective portions of the first adhesive layer 130 and the second adhesive layer 140 may be selectively exposed by removal of the first release liner 161 and the second release liner 162.

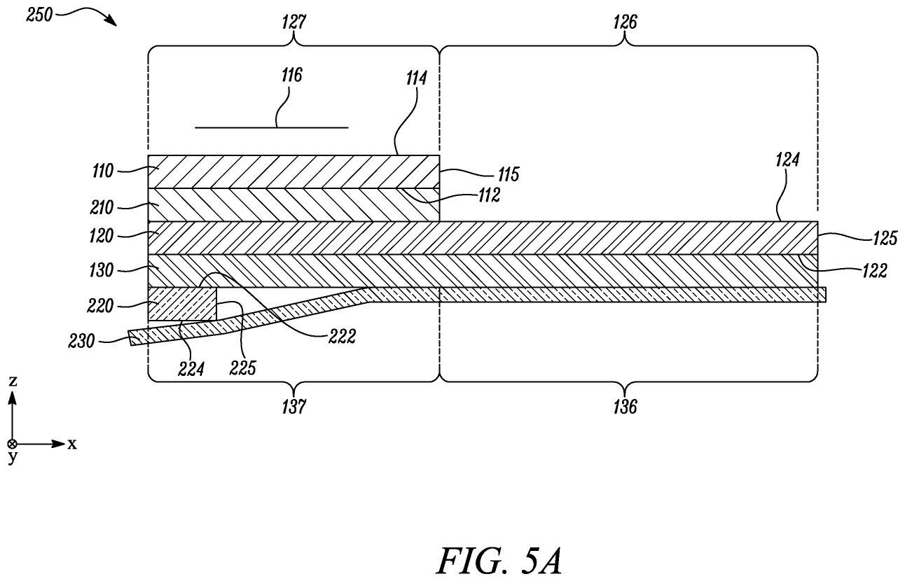

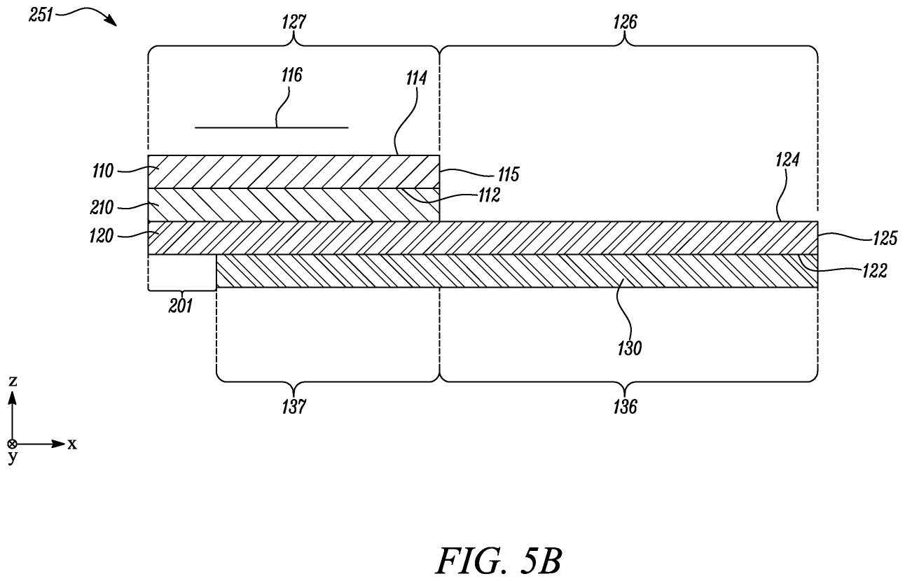

FIG. 5A illustrates a schematic cross-sectional view of a cover dressing 250 according to another embodiment of the present disclosure. The cover dressing 250 is similar to the cover dressing 100 of FIG. 2A, with like elements designated by like reference characters. However, the cover dressing 250 has a different configuration of the first adhesive layer 130 than that of the cover dressing 100 of FIG. 2A. Further, the cover dressing 250 omits the second adhesive layer 140 of the cover dressing 100 of FIG. 2A. Further, the first adhesive layer 130 is disposed on the cover film 120, such that the cover film 120 is disposed between the backing 110 and the first adhesive layer 130 along a thickness (i.e., the z-axis) of the cover dressing 250.

In the illustrated embodiment of FIG. 5A, the cover dressing 250 includes a tie layer 210 at least partially disposed between the first major surface 112 of the backing 110 and the second major surface 124 of the cover film 120. The tie layer 210 attaches the cover film 120 to the backing 110. It may be noted that the tie layer 210 is optional and may be omitted from the cover dressing 250, for example, if the cover film 120 is attached to the backing 110 by mechanical means, such as by stitching.

In the illustrated embodiment of FIG. 5A, the first adhesive layer 130 is at least partially disposed on the first major surface 122 of the cover film 120. Specifically, in the illustrated embodiment of FIG. 5A, the first adhesive layer 130 is disposed on the first major surface 122 of the cover film 120 opposite to the backing 110. Therefore, the backing 110 is free of the first adhesive layer 130.

As discussed above, the first adhesive layer 130 defines the first adhesive portion 136 extending beyond the backing perimeter 115 in the dressing plane 116. The first adhesive portion 136 of the first adhesive layer 130 is configured to at least partially bond with the skin of the patient (e.g., the skin 20 of the patient 22 of FIG. 1). In the illustrated embodiment of FIG. 5A, the first adhesive layer 130 further defines a second adhesive portion 137 at least partially coextensive with the backing 110 in the dressing plane 116. In some embodiments, the second adhesive portion 137 is configured to at least partially bond with the medical device 10 (shown in FIG. 1).

In the illustrated embodiment of FIG. 5A, the cover dressing 250 further includes a tab 220. In the illustrated embodiment of FIG. 5A, the tab 220 is disposed on the first adhesive layer 130, such that the tab 220 is spaced apart from the first adhesive portion 136 in the dressing plane 116. In the illustrated embodiment of FIG. 5A, the tab 220 is disposed on the second adhesive portion 137 of the first adhesive layer 130 and spaced apart from the first adhesive portion 136 in the dressing plane 116.

The tab 220 may include a first major surface 222 and a second major surface 224 opposing the first major surface 222 of the tab 220. In the illustrated embodiment of FIG. 5A, the first major surface 222 of the tab 220 faces the first major surface 112 of the cover film 120. The tab 220 may further include a tab perimeter 225 surrounding the first major surface 222 and the second major surface 224 of the tab 220. The backing 110 may extend beyond the tab perimeter 225, such that the backing perimeter 115 is at least partially spaced apart from the tab perimeter 225 in the dressing plane 116. Further, in the illustrated embodiment of FIG. 5A, the tab perimeter 225 is partially aligned with the backing perimeter 115 in the dressing plane 116. In some embodiments, an area of the second major surface 224 of the tab 220 is at most 30% of an area of the second major surface 114 of the backing 110.

The cover dressing 250 may further include a release liner 230. In the illustrated embodiment of FIG. 5A, the release liner 230 is at least partially and removably adhered to the first adhesive layer 130. A removal of the release liner 230 may expose at least a portion of the first adhesive layer 130.

FIG. 5B illustrates a schematic cross-sectional view of a cover dressing 251 according to another embodiment of the present disclosure. The cover dressing 251 is substantially similar to the cover dressing 250 of FIG. 5A, with like elements designated by like reference characters. However, the cover dressing 251 has a different configuration of the first adhesive layer 130 than that of the cover dressing 250.

Specifically, in the illustrated embodiment of FIG. 5B, the first major surface 122 of the cover film 120 includes an adhesive-free region 201 extending beyond the first adhesive layer 130. In other words, in the illustrated embodiment of FIG. 5B, the first major surface 122 of the cover film 120 defines the adhesive-free region 201 free of the first adhesive layer 130. The adhesive-free region 201 may enable omission of the tab 220 (shown in FIG. 5A) from the cover dressing 251. As a result, in the illustrated embodiment of FIG. 5B, the cover dressing 251 omits the tab 220.

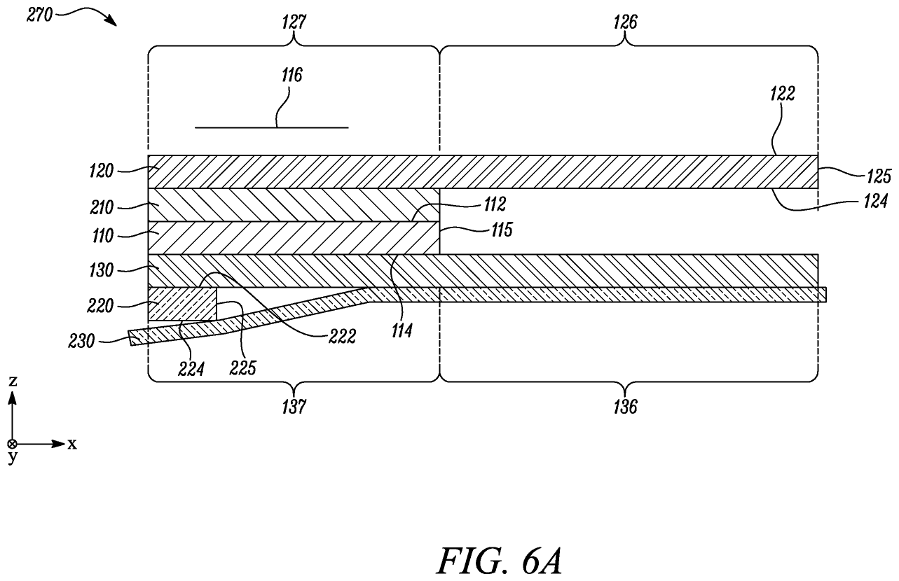

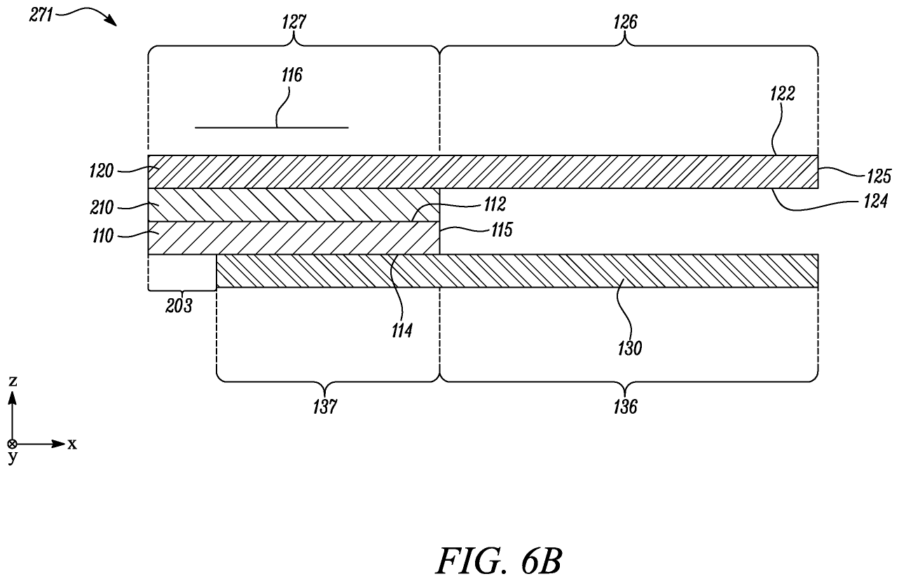

FIG. 6A illustrates a schematic cross-sectional view of a cover dressing 270 according to another embodiment of the present disclosure. The cover dressing 270 is similar to the cover dressing 250 of FIG. 5A, with like elements designated by like reference characters. However, the cover dressing 270 has a different configuration of the first adhesive layer 130 than that of the cover dressing 250. Further, the first adhesive layer 130 is at least partially disposed on the backing 110, such that the backing 110 is disposed between the cover film 120 and the first adhesive layer 130 along a thickness (i.e., the z-axis) of the cover dressing 270.

In the illustrated embodiment of FIG. 6A, the first adhesive layer 130 is at least partially disposed on the second major surface 114 of the backing 110. As discussed above, the first adhesive layer 130 defines the first adhesive portion 136 extending beyond the backing perimeter 115 in the dressing plane 116. In the illustrated embodiment of FIG. 6A, the first adhesive layer 130 further defines the second adhesive portion 137 at least partially coextensive with the backing 110.

In the illustrated embodiment of FIG. 6A, the cover dressing 270 further includes the tab 220. In the illustrated embodiment of FIG. 6A, the first major surface 222 of the tab 220 faces the second major surface 114 of the backing 110. The cover dressing 270 may optionally further include the release liner 230.

FIG. 6B illustrates a schematic cross-sectional view of a cover dressing 271 according to another embodiment of the present disclosure. The cover dressing 271 is similar to the cover dressing 270 of FIG. 6A, with like elements designated by like reference characters. However, the cover dressing 271 has a different configuration of the first adhesive layer 130 than that of the cover dressing 270.

Specifically, in the illustrated embodiment of FIG. 6B, the second major surface 114 of the backing 110 includes an adhesive-free region 203 extending beyond the first adhesive layer 130. In other words, in the illustrated embodiment of FIG. 6B, the second major surface 114 of the backing 110 defines the adhesive-free region 203 free of the first adhesive layer 130. The adhesive-free region 203 may enable omission of the tab 220 (shown in FIG. 6A) from the cover dressing 271. Therefore, in the illustrated embodiment of FIG. 6B, the cover dressing 271 omits the tab 220.

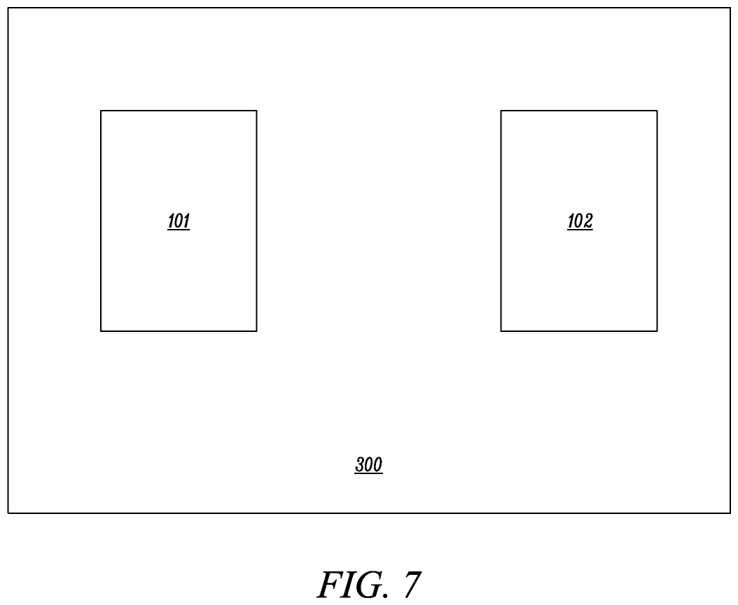

FIG. 7 illustrates a schematic block diagram of a kit 300 for use with a medical device insertable through a skin of a patient at an insertion site according to an embodiment of the present disclosure.

The kit 300 includes a first cover dressing 101 and a second cover dressing 102. Each of the first cover dressing 101 and the second cover dressing 102 may be equivalent to the cover dressing 100 of FIG. 2A, the cover dressing 170 of FIG. 3, the cover dressing 200 of FIG. 4, the cover dressing 250 of FIG. 5A, the cover dressing 251 of FIG. 5B, the cover dressing 270 of FIG. 6A, or the cover dressing 271 of FIG. 6B. In some embodiments, each of the first cover dressing 101 and the second cover dressing 102 may be identical to each other. In some other embodiments, the first cover dressing 101 and the second cover dressing 102 may be different from each other. For example, in some embodiments, the first cover dressing 101 may be equivalent to the cover dressing 100 of FIG. 2A and the second cover dressing 102 may be equivalent to the cover dressing 200 of FIG. 3.

Referring to FIGS. 2A to 4, in some embodiments, each of the first cover dressing 101 and the second cover dressing 102 includes the backing 110 including the first major surface 112, the second major surface 114 opposing the first major surface 112, and the backing perimeter 115 surrounding the first major surface 112 and the second major surface 114 of the backing 110. The first major surface 112 of the backing 110 defines the dressing plane 116.

Further, in some embodiments, each of the first cover dressing 101 and the second cover dressing 102 includes the cover film 120 including the first major surface 122, the second major surface 124 opposing the first major surface 122 and facing the first major surface 112 of the backing 110, and the film perimeter 125 surrounding the first major surface 122 and the second major surface 124 of the cover film 120. The cover film 120 defines the first film portion 126 extending beyond the backing perimeter 115 and the second film portion 127 at least partially coextensive with the backing 110, such that the film perimeter 125 corresponding to the first film portion 126 is at least partially spaced apart from the backing perimeter 115 in the dressing plane 116.

Further, in some embodiments, each of the first cover dressing 101 and the second cover dressing 102 includes the first adhesive layer 130 partially disposed between the first major surface 112 of the backing 110 and the second major surface 124 of the cover film 120 and including the first adhesive. The first adhesive layer 130 is further at least partially disposed on the second major surface 124 of the cover film 120 corresponding to the first film portion 126. The first adhesive layer 130 is configured to at least partially bond with the skin 20 of the patient 22 (shown in FIG. 1).

Further, in some embodiments, each of the first cover dressing 101 and the second cover dressing 102 includes the second adhesive layer 140 at least partially disposed on the second major surface 114 of the backing 110 The second adhesive layer 140 is configured to at least partially bond with the medical device 10 (shown in FIG. 1).

In some embodiments, the kit 300 may further include gloves, a sterilizing material, or a cloth or other absorbent materials. The kit 300 may be available to a clinician for use in a sterilized package. The first cover dressing 101 and the second cover dressing 102 may be readily available to the clinician for application on the skin of the patient.

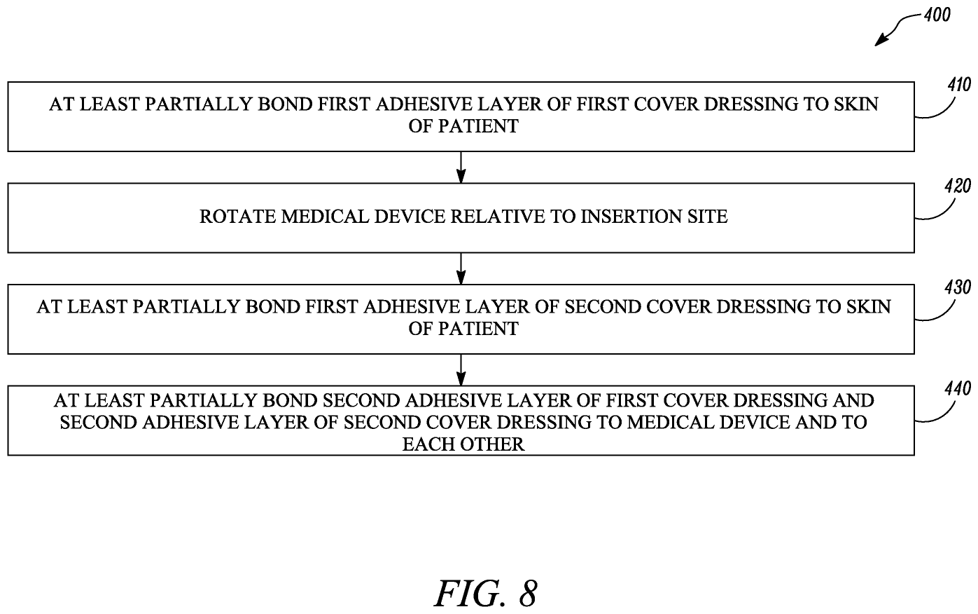

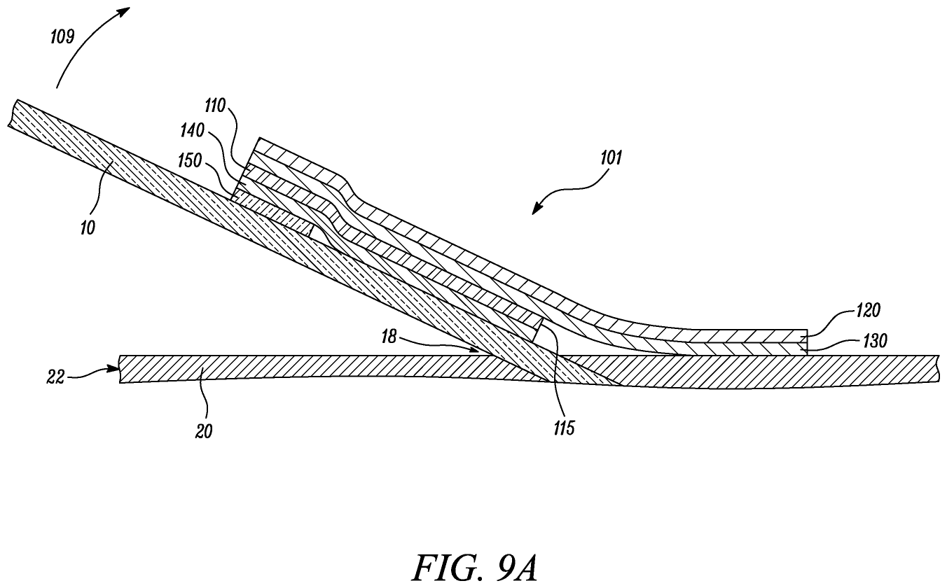

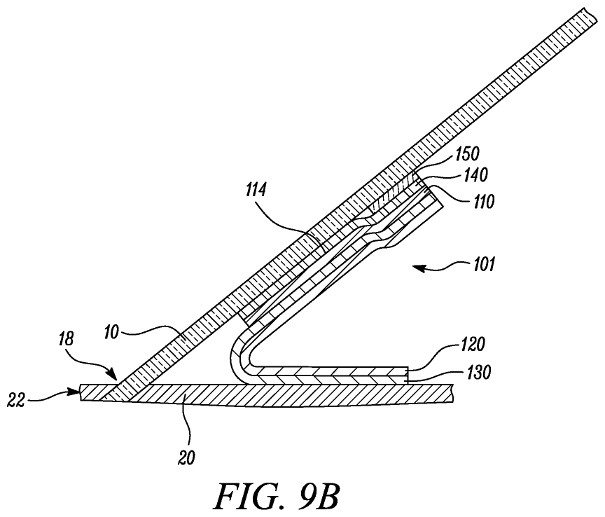

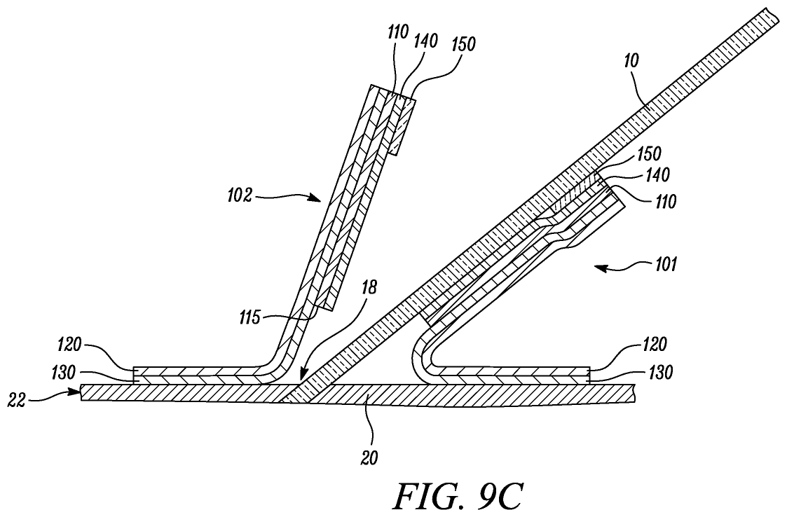

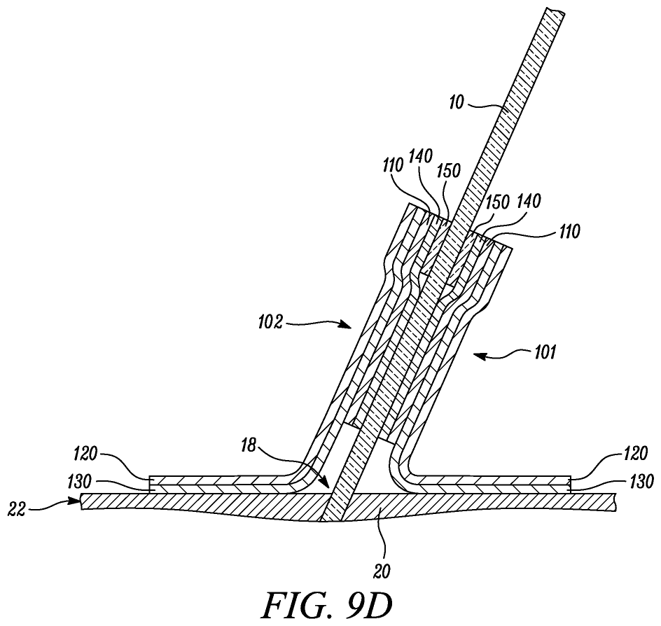

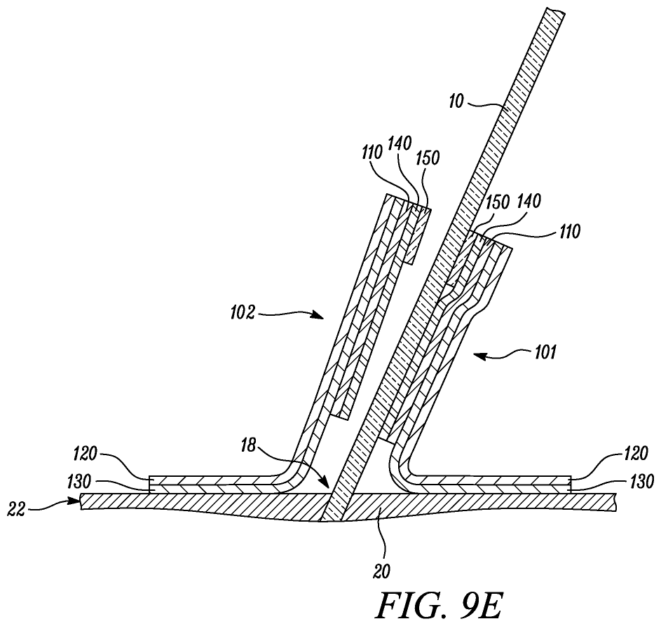

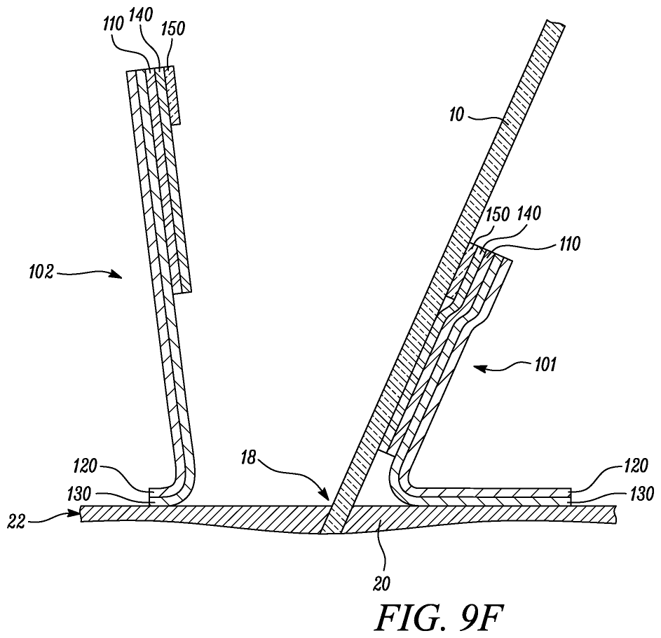

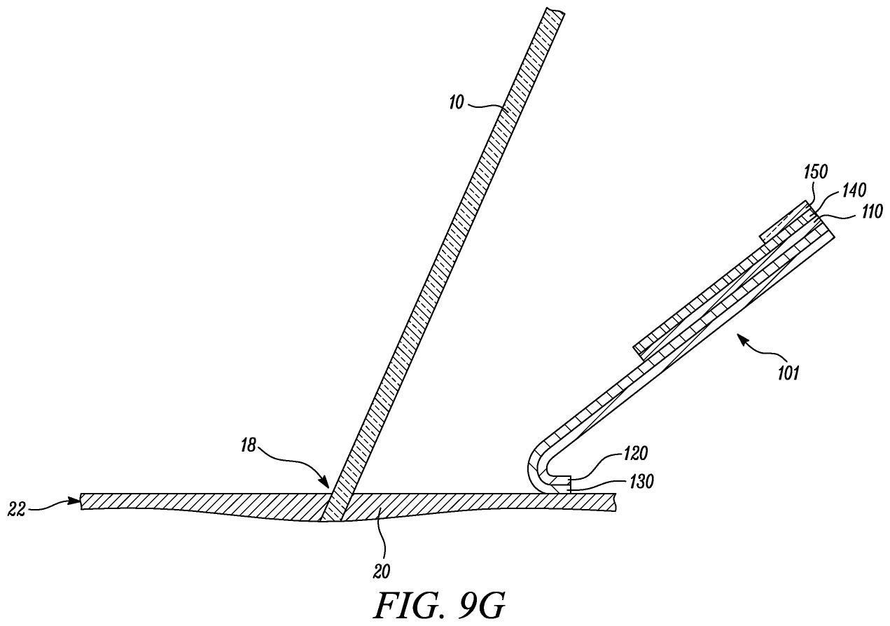

FIG. 8 illustrates a flowchart of a method 400 of using the kit 300 of FIG. 7 according to an embodiment of the present disclosure. The method 400 is schematically illustrated in FIGS. 9A-9G. The method 400 will be discussed with reference to FIGS. 8 and 9A-9G.

At step 410, the method 400 includes at least partially bonding the first adhesive layer of the first cover dressing to the skin of the patient, such that at least a portion of the backing perimeter of the first cover dressing is disposed adjacent to the insertion site. For example, as shown in FIG. 9A, the method 400 may include at least partially bonding the first adhesive layer 130 of the first cover dressing 101 to the skin 20 of the patient 22, such that at least a portion of the backing perimeter 115 of the first cover dressing 101 is disposed adjacent to the insertion site 18. The method 400 may further include at least partially bonding the second adhesive layer 140 of the first cover dressing 101 to the medical device 10.

At step 420, the method 400 further includes rotating the medical device relative to the insertion site, such that the first cover dressing is folded and the second major surface of the backing faces away from the skin. For example, as shown in FIGS. 9A and 9B, the method 400 may include rotating the medical device 10 relative to the insertion site 18 (e.g., in a direction 109 shown in FIG. 9A), such that the first cover dressing 101 is folded and the second major surface 114 of the backing 110 faces away from the skin 20 (shown in FIG. 9B).

At step 430, the method 400 further includes at least partially bonding the first adhesive layer of the second cover dressing to the skin of the patient, such that at least a portion of the backing perimeter of the second cover dressing is disposed adjacent to the insertion site, and the insertion site is together covered by the first cover dressing and the second cover dressing. For example, as shown in FIG. 9C, the method 400 may include at least partially bonding the first adhesive layer 130 of the second cover dressing 102 to the skin 20 of the patient 22, such that at least a portion of the backing perimeter 115 of the second cover dressing 102 is disposed adjacent to the insertion site 18, and the insertion site 18 is together covered by the first cover dressing 101 and the second cover dressing 102. It may be noted that the insertion site 18 may be surrounded by the cover film 120 of the first cover dressing 101 and the cover film 120 of the second cover dressing 102.

At step 440, the method 400 further includes at least partially bonding the second adhesive layer of the first cover dressing and the second adhesive layer of the second cover dressing to the medical device and to each other, such that the medical device is at least partially disposed between and secured to the first cover dressing and the second cover dressing. For example, as shown in FIG. 9D, the method 400 may include at least partially bonding the second adhesive layer 140 of the first cover dressing 101 and the second adhesive layer 140 of the second cover dressing 102 to the medical device 10 and to each other, such that the medical device 10 is at least partially disposed between and secured to the first cover dressing 101 and the second cover dressing 102. The first cover dressing 101 and the second cover dressing 102 may be at least partially wrapped around the medical device 10, such that the second adhesive layer 140 of the first cover dressing 101 and the second adhesive layer 140 of the second cover dressing 102 can be bonded to each other.

In some embodiments, the method 400 further includes removing the second adhesive layer of the second cover dressing from the medical device and the second adhesive layer of the first cover dressing. For example, as shown in FIG. 9E, the method 400 may include removing the second adhesive layer 140 of the second cover dressing 102 from the medical device 10 and the second adhesive layer 140 of the first cover dressing 101. The second adhesive of the second adhesive layer 140 may facilitate removal of the second adhesive layer 140 of the second cover dressing 102 from the medical device 10 and the second adhesive layer 140 of the first cover dressing 101.

In some embodiments, the method 400 further includes removing the first adhesive layer of the second cover dressing from the skin of the patient. For example, as shown in FIG. 9F, the method 400 may include removing the first adhesive layer 130 of the second cover dressing 102 from the skin 20 of the patient 22.

In some embodiments, the method 400 further includes removing the second adhesive layer of the second cover dressing from the medical device. For example, as shown in FIG. 9F, the method 400 may include removing the second adhesive layer 140 of the second cover dressing 102 from the medical device 10.

In some embodiments, the method 400 further includes removing the first adhesive layer of the first cover dressing from the skin of the patient. For example, as shown in FIG. 9G, the method 400 may include removing the first adhesive layer 130 of the first cover dressing 101 from the skin 20 of the patient 22.

The method 400 may enable the clinician to use the kit 300 to apply the first cover dressing 101 and the second cover dressing 102 to the skin 20 of the patient 22 (shown in FIG. 1). The first cover dressing 101 and the second cover dressing 102 may therefore be used to cover the insertion site 18, and protect the insertion site 18 from dirt, bacteria, and moisture, thereby decreasing a risk of infection at the insertion site 18. The method 400 may further enable the clinician to use the kit 300 to remove the first cover dressing 101 and the second cover dressing 102 from the skin 20 of the patient 22.

Unless otherwise indicated, all numbers expressing feature sizes, amounts, and physical properties used in the specification and claims are to be understood as being modified by the term “about”. Accordingly, unless indicated to the contrary, the numerical parameters set forth in the foregoing specification and attached claims are approximations that can vary depending upon the desired properties sought to be obtained by those skilled in the art utilizing the teachings disclosed herein.

Although specific embodiments have been illustrated and described herein, it will be appreciated by those of ordinary skill in the art that a variety of alternate and/or equivalent implementations can be substituted for the specific embodiments shown and described without departing from the scope of the present disclosure. This application is intended to cover any adaptations or variations of the specific embodiments discussed herein. Therefore, it is intended that this disclosure be limited only by the claims and the equivalents thereof.

What is claimed is:

1. A cover dressing, the cover dressing comprising:
a backing comprising a first major surface, a second major surface opposing the first major surface, and a backing perimeter surrounding the first major surface and the second major surface of the backing, the first major surface of the backing defining a dressing plane;
a cover film comprising a first major surface, a second major surface opposing the first major surface and facing the first major surface of the backing, and a film perimeter surrounding the first major surface and the second major surface of the cover film, wherein the cover film defines a first film portion extending beyond the backing perimeter and a second film portion at least partially coextensive with the backing, such that the film perimeter corresponding to the first film portion is at least partially spaced apart from the backing perimeter in the dressing plane; and
a first adhesive layer at least partially disposed on at least one of the backing and the cover film, wherein the first adhesive layer defines a first adhesive portion extending beyond the backing perimeter in the dressing plane, and wherein the first adhesive portion of the first adhesive layer is configured to at least partially bond with a skin of a patient.

2. The cover dressing of claim 1, wherein first adhesive layer is at least partially disposed on the first major surface of the cover film.

3. The cover dressing of claim 1, wherein the first adhesive layer further defines a second adhesive portion at least partially coextensive with the backing in the dressing plane, and wherein the second adhesive portion is configured to at least partially bond with a medical device.

4. The cover dressing of claim 1, further comprising a tie layer at least partially disposed between the first major surface of the backing and the second major surface of the cover film, and wherein the tie layer attaches the cover film to the backing.

5. The cover dressing of claim 1, wherein the first adhesive layer is at least partially disposed on the second major surface of the backing

6. The cover dressing of claim 1, further comprising a tab disposed on the first adhesive layer, such that the tab is spaced apart from the first adhesive portion in the dressing plane, the tab comprising a first major surface, a second major surface opposing the first major surface of the tab, and a tab perimeter surrounding the first major surface and the second major surface of the tab, wherein the backing extends beyond the tab perimeter, such that the backing perimeter is at least partially spaced apart from the tab perimeter in the dressing plane.

7. The cover dressing of claim 6, wherein the tab perimeter is partially aligned with the backing perimeter in the dressing plane.

8. The cover dressing of claim 6, wherein an area of the second major surface of the tab is at most 30% of an area of the second major surface of the backing.

9. The cover dressing of claim 1, further comprising a release liner at least partially and removably adhered to the first adhesive layer, and wherein a removal of the release liner exposes at least a portion of the first adhesive layer.

10. The cover dressing of claim 1, wherein the first adhesive layer is partially disposed between the first major surface of the backing and the second major surface of the cover film, such that the first adhesive portion is at least partially coextensive with the first film portion in the dressing plane.

11. The cover dressing of claim 1, further comprising a second adhesive layer at least partially disposed on the second major surface of the backing, wherein the second adhesive layer is configured to at least partially bond with a medical device.

12. The cover dressing of claim 11, wherein the first adhesive layer is coextensive with the second major surface of the cover film.

13. The cover dressing of claim 11, wherein the second adhesive layer is coextensive with the second major surface of the backing.

14. The cover dressing of claim 11, wherein the second adhesive layer comprises a silicone adhesive.

15. The cover dressing of claim 11, wherein the second major surface of the backing comprises an adhesive-free region extending beyond the second adhesive layer.

16. The cover dressing of claim 11, further comprising a release liner at least partially and removably adhered to each of the first adhesive layer and the second adhesive layer, and wherein a removal of the release liner exposes at least respective portions of the first adhesive layer and the second adhesive layer.

17. The cover dressing of claim 11, further comprising:
a first release liner at least partially and removably adhered to the first adhesive layer, wherein a removal of the first release liner exposes at least a portion of the first adhesive layer; and
a second release liner at least partially and removably adhered to the second adhesive layer, wherein a removal of the second release liner exposes at least a portion of the second adhesive layer.

18. The cover dressing of claim 11, further comprising a tab comprising a first major surface facing the second major surface of the backing, a second major surface opposing the first major surface of the tab, and a tab perimeter surrounding the first major surface and the second major surface of the tab, wherein each of the backing and the second adhesive layer extends beyond the tab perimeter, such that the backing perimeter is at least partially spaced apart from the tab perimeter in the dressing plane, and wherein the second adhesive layer is partially disposed between the second major surface of the backing and the first major surface of the tab.

19. The cover dressing of claim 18, wherein the tab perimeter is partially aligned with the backing perimeter in the dressing plane.

20. The cover dressing of claim 18, wherein each of the backing, the cover film, the first adhesive layer, the second adhesive layer, and the tab comprises a respective first transverse edge and a respective second transverse edge opposite to the respective first transverse edge, and wherein the respective first transverse edges of the backing, the cover film, the first adhesive layer, the second adhesive layer, and the tab are aligned in the dressing plane.

21. The cover dressing of claim 1, wherein the second major surface of the backing comprises an adhesive-free region extending beyond the first adhesive layer.

22. The cover dressing of claim 1, wherein the first major surface of the cover film comprises an adhesive-free region extending beyond the first adhesive layer.

23. The cover dressing of claim 1, wherein the backing perimeter is partially aligned with the film perimeter in the dressing plane.

24. The cover dressing of claim 1, wherein an area of the first major surface of the backing is at most 60% of an area of the second major surface of the cover film.

25. The cover dressing of claim 1, wherein the cover film is optically transparent.

26. The cover dressing of claim 1, wherein the backing comprises a foam or a nonwoven material.

27. The cover dressing of claim 1, wherein the first adhesive layer comprises an antimicrobial agent.

28. The cover dressing of claim 1, wherein each of the backing and the cover film is continuous in the dressing plane and is devoid of any through-openings.

29. A kit for use with a medical device insertable through a skin of a patient at an insertion site, the kit comprising a pair of cover dressings according to claim 1.

30. A kit for use with a medical device insertable through a skin of a patient at an insertion site, the kit comprising a first cover dressing and a second cover dressing, each of the first cover dressing and the second cover dressing comprising:
a backing comprising a first major surface, a second major surface opposing the first major surface, and a backing perimeter surrounding the first major surface and the second major surface of the backing, the first major surface of the backing defining a dressing plane;
a cover film comprising a first major surface, a second major surface opposing the first major surface and facing the first major surface of the backing, and a film perimeter surrounding the first major surface and the second major surface of the cover film, wherein the cover film defines a first film portion extending beyond the backing perimeter and a second film portion at least partially coextensive with the backing, such that the film perimeter corresponding to the first film portion is at least partially spaced apart from the backing perimeter in the dressing plane;
a first adhesive layer partially disposed between the first major surface of the backing and the second major surface of the cover film, wherein the first adhesive layer is further at least partially disposed on the second major surface of the cover film corresponding to the first film portion, and wherein the first adhesive layer is configured to at least partially bond with the skin of the patient; and
a second adhesive layer at least partially disposed on the second major surface of the backing, wherein the second adhesive layer is configured to at least partially bond with the medical device.

31. A method of using the kit of claim 30, the method comprising:
at least partially bonding the first adhesive layer of the first cover dressing to the skin of the patient, such that at least a portion of the backing perimeter of the first cover dressing is disposed adjacent to the insertion site;
rotating the medical device relative to the insertion site, such that the first cover dressing is folded and the second major surface of the backing faces away from the skin;
at least partially bonding the first adhesive layer of the second cover dressing to the skin of the patient, such that at least a portion of the backing perimeter of the second cover dressing is disposed adjacent to the insertion site, and the insertion site is together covered by the first cover dressing and the second cover dressing; and
at least partially bonding the second adhesive layer of the first cover dressing and the second adhesive layer of the second cover dressing to the medical device and to each other, such that the medical device is at least partially disposed between and secured to the first cover dressing and the second cover dressing.

32. The method of claim 31, further comprising:
removing the second adhesive layer of the second cover dressing from the medical device and the second adhesive layer of the first cover dressing;
removing the first adhesive layer of the second cover dressing from the skin of the patient;
removing the second adhesive layer of the second cover dressing from the medical device; and
removing the first adhesive layer of the first cover dressing from the skin of the patient.
